(19) United States
(12) Patent Application Publication
(10) Pub. No.: US 2026/0134947 A1
(43) Pub. Date: May 14, 2026

(54) METHOD AND SYSTEM FOR PREDICTING IMMUNE RESPONSE AND COMPUTER READABLE MEDIUM THEREOF

(71) Applicant: National Yang Ming Chiao Tung University, Hsinchu City (TW)

(72) Inventors: Tai-Ming Ko, Hsinchu City (TW); Jing-Rong Wang, Tainan City (TW)

(73) Assignee: National Yang Ming Chiao Tung University, Hsinchu City (TW)

(21) Appl. No.: 18/946,881

(22) Filed: Nov. 13, 2024

(51) Int. Cl. G16B 40/20; G16B 20/00; G16B 25/10

(52) U.S. Cl. CPC G16B 40/20; G16B 20/00; G16B 25/10

(57) ABSTRACT

A method for predicting an immune response includes: providing a transcriptomic data of a single cell to a deep learning model to create a training data set; and building an immune response prediction model based on the training data set. A system for predicting the immune response includes: a genetic feature extraction unit used to provide the transcriptomic data of the single cell to the deep learning model to create the training data set; and a model building and optimization unit used to build the immune response prediction model based on the training data set. A computer readable medium storing a computer executable code which, when being executed, causes the method to be implemented.

### BACKGROUND OF THE INVENTION

### 1. Field of the Invention

The present disclosure relates to single-cell transcriptomic data analysis, and more particularly to method and system for predicting an immune response and computer readable medium thereof.

### 2. Description of the Prior Art

In the realm of drug development of the biopharmaceuticals, i.e., biologics (or called biological agents) and biosimilars, there are two significant problems. Expanding the number of treated cases for biological agents is the first challenge. In particular, biological agents, e.g., antibody drugs or biological disease-modifying antirheumatic drugs (bDMARDs), offer targeted mechanisms to regulate immune responses. However, pharmaceutical companies face hurdles in expanding their use to new indications or disease conditions due to the high costs and complexities involved in testing these new applications. Reducing costs in new drug development through patient stratification is the second challenge. The cost of developing new drugs, especially bDMARDs, is exceptionally high. A major portion of this cost is attributed to clinical trials, which often face challenges in patient recruitment and retention, and identifying the appropriate patient groups for specific treatments.

Furthermore, patients with autoimmune diseases respond differently to treatment of biological agents, and the duration of the treatment (approximately from 6 months to 1 year) usually takes longer than that of the general drugs, which may delay effective treatment. In order to prevent the patients with autoimmune diseases from suffering during the duration of the treatment, helping them to find the appropriate treatment strategy as early as possible is needed. In previous studies, there are multiple ways to predict the effectiveness of the drug, e.g., analyzing the blood samples of the patients with rheumatoid arthritis (RA) before and after the treatment by qPCR.

However, the predictability of treatment outcomes is growing more challenging due to the intricate variables of patient heterogeneity, diverse blood immune profiles, and active pathogenic pathways. Hence, the ongoing medical challenges encompass the inability to predict, prevent, and cure the diseases; specifically highlighting the lack of predictive capabilities to determine which patients will respond or face toxic risks from specific treatments.

Based on the above, there is an unmet need in the art to prevent or alleviate clinically significant damage by early diagnosis and prompt therapy initiation for rapidly predicting biologics response of the patients, assessing the suitability of a given biologics for the patients.

### SUMMARY OF THE INVENTION

A method for predicting an immune response includes a genetic feature extraction unit providing a transcriptomic data of a single cell to a deep learning model to create a training data set; and a model building and optimization unit building an immune response prediction model based on the training data set.

A system for predicting an immune response includes: a genetic feature extraction unit used to provide a transcriptomic data of a single cell to a deep learning model to create a training data set; and a model building and optimization unit used to build an immune response prediction model based on the training data set.

A computer readable medium storing a computer executable code which, when being executed, causes the method to be implemented.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating an exemplifying structure of the system for predicting an immune response in accordance with embodiments of the present disclosure.

FIG. 2 is a schematic diagram illustrating an exemplifying structure of the method for predicting an immune response in accordance with embodiments of the present disclosure.

FIG. 3A is a schematic diagram illustrating an exemplifying workflow of constructing an immune response prediction model in accordance with embodiments of the present disclosure.

FIG. 3B is scatter graph and line graph illustrating the prediction performances of the immune response prediction model in accordance with embodiments of the present disclosure.

FIG. 3C is a stacked bar graph illustrating the percentages of identical differentially expressed genes (DEGs) and pathways by comparing real transcriptomic data and baseline data, and model-predicted transcriptomic data and real baseline data in accordance with embodiments of the present disclosure.

FIG. 3D is a visualized enrichment plots illustrating the expected down-regulated gene set of anti-CTLA-4-perturbed pathways via a gene set enrichment analysis (GSEA) in accordance with embodiments of the present disclosure.

FIG. 4A is scatter graph and line graph illustrating the prediction performances of the immune response prediction model in accordance with embodiments of the present disclosure.

FIG. 4B is scatter graph and line graph illustrating the prediction performances of the immune response prediction model in accordance with embodiments of the present disclosure.

FIG. 5A is a schematic diagram illustrating an exemplifying flowchart of performing immune response prediction via an immune response prediction model in accordance with embodiments of the present disclosure.

FIG. 5B is a visualized enrichment plots illustrating the distinguished therapeutic pathways between responders (Rs) and non-responders (NRs) with reverse regulation of each other in real data via a gene set enrichment analysis (GSEA) in accordance with embodiments of the present disclosure.

FIG. 5C is a visualized enrichment plots illustrating the distinguished therapeutic pathways between responders (Rs) and non-responders (NRs) with reverse regulation of each other in predicted data via a gene set enrichment analysis (GSEA) in accordance with embodiments of the present disclosure.

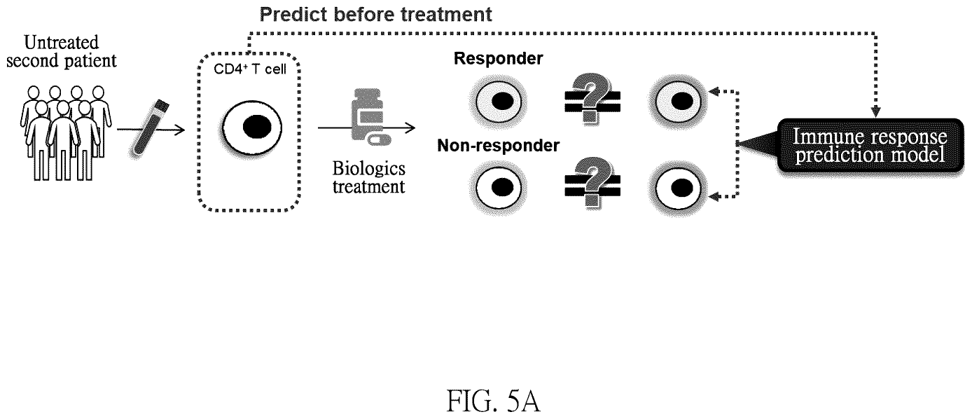

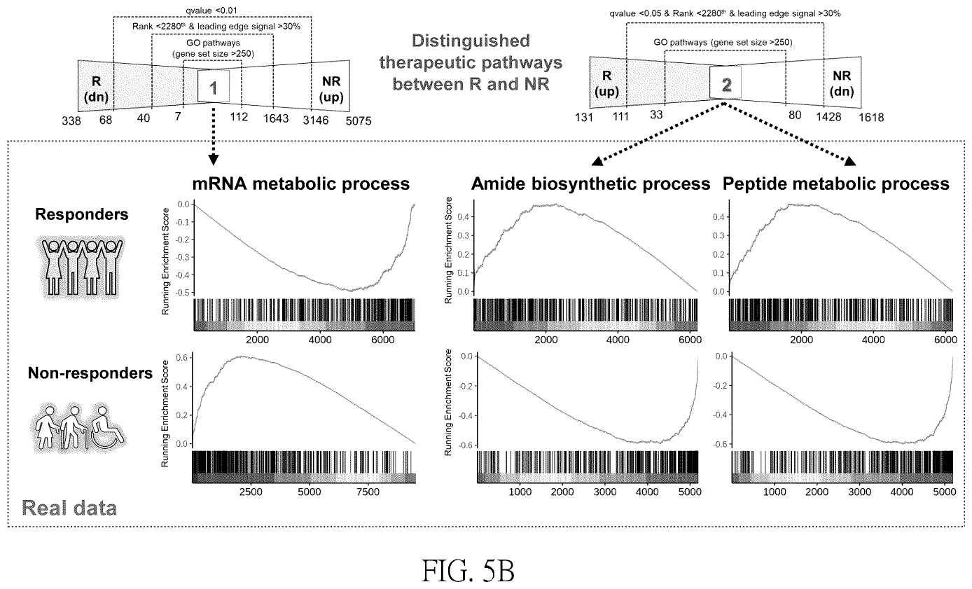

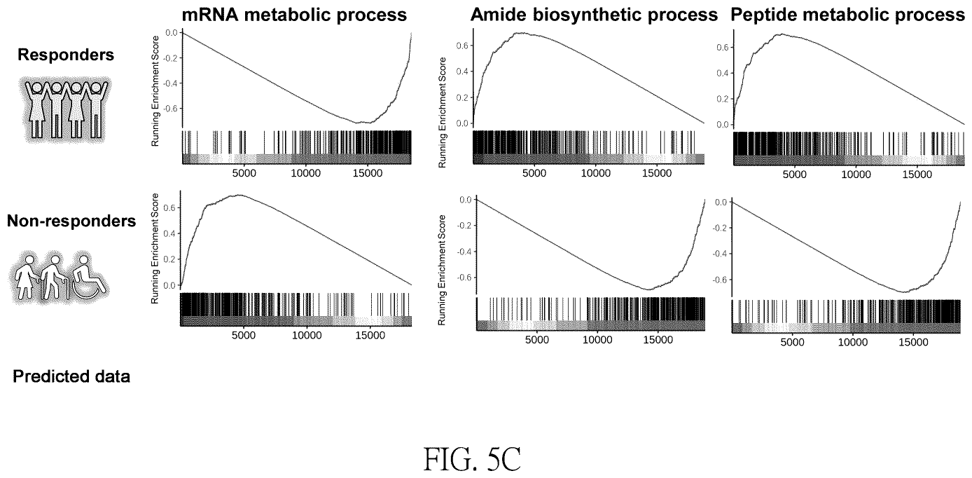

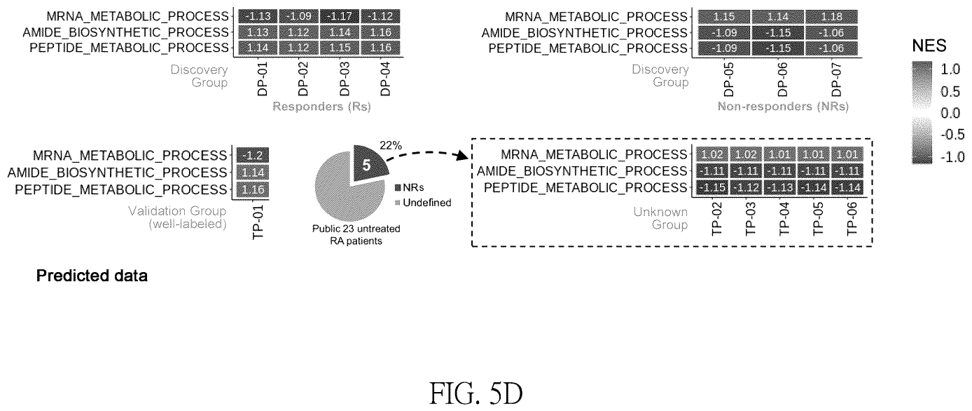

The upper part of FIG. 5D are heat maps of normalized enrichment score (NES) illustrating the target pathway perturbations of the Rs and NRs predicted by the immune response prediction model in accordance with embodiments of the present disclosure.

The bottom left of FIG. 5D is a heat map illustrating that the patient labeled a responder shows the target pathway perturbations equivalent to the Rs in predicted data in accordance with embodiments of the present disclosure.

The bottom center and bottom right of FIG. 5D is a pie chart and a heat map illustrating that the untreated RA patients without therapeutic outcomes are stratified by their NES of biologics treatment perturbed target pathways predicted by the immune response prediction model in accordance with embodiments of the present disclosure.

### DETAILED DESCRIPTION

The following embodiments are provided to illustrate the present disclosure in detail. A person having ordinary skill in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

The proportional relationships, structures, sizes and other features shown in accompanying drawings of this disclosure are only used to illustrate embodiments describe herein, such that those with ordinary skill in the art can read and understand the present disclosure therefrom, of which are not intended to limit the scope of this disclosure. Any changes, modifications, or adjustments of said features, without affecting the designed purposes and effects of the present disclosure, should all fall within the scope of the technical content of this disclosure.

As used herein, sequential terms such as “first,” “second,” etc., are only cited in convenience of describing or distinguishing limitations such as elements, components, structures, regions, units, modules, models, parts, devices, systems, etc. from one another, which are not intended to limit the scope of this disclosure, nor to limit spatial sequences between such limitations. Further, unless otherwise specified, wordings in singular forms such as “a,” “an” and “the” also pertain to plural forms, and wordings such as “or” and “and/or” may be used interchangeably.

As used herein, the terms “individual,” “participant,” and “patient” may be interchangeable and refer to an animal, e.g., a mammal including the human species. The term “patient” is intended to refer to both the male and female gender unless one gender is specifically indicated.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other elements, components, structures, regions, units, modules, models, parts, devices, systems, steps, or connections, etc., and should not exclude other limitations.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the terms “one or more” and “at least one” may have the same meaning and include one, two, three, or more.

Unless otherwise specified, terms “biologics,” “biological agents,” and “biologic agents” used herein may be used interchangeably.

Unless otherwise specified, terms “treatment” and “therapy” used herein may be used interchangeably.

In at least one embodiment of the present disclosure, the single cell may include an immune cell. In some embodiments, the immune cell may include a monocyte, a lymphocyte, and/or a granulocyte. In some embodiments of the present disclosure, the lymphocyte may include a T cell.

In at least one embodiment of the present disclosure, the transcriptomic data provided to the deep learning model may include a first transcriptomic data and a second transcriptomic data, and the immune response prediction model may be built by training a variational autoencoder of the deep learning model with the first transcriptomic data and the second transcriptomic data.

In at least one embodiment of the present disclosure, the method of the present disclosure may further include: providing a third transcriptomic data of the single cell to the deep learning model to create a testing data set; and importing the third transcriptomic data to the immune response prediction model to predict the immune response.

In at least one embodiment of the present disclosure, the providing the transcriptomic data of the single cell to the deep learning model to create the training data set may include: using a first genetic feature of the first transcriptomic data and a second genetic feature of the second transcriptomic data as the training data set by data preprocessing and data transformation processing; and the providing the third transcriptomic data of the single cell to the deep learning model to create the testing data set may include: using a third genetic feature of the third transcriptomic data as the testing data set.

In at least one embodiment of the present disclosure, the data preprocessing may include a transcriptomic identification of the single cell, and the data transformation processing may include a transformation operation of the genetic signature.

In at least one embodiment of the present disclosure, the first transcriptomic data is obtained from a first blood sample of a first patient with autoimmune disease before administration of a biologics; the second transcriptomic data is obtained from a second blood sample of the first patient with autoimmune disease after administration of the biologics; and the third transcriptomic data is obtained from a third blood sample of the second patient with autoimmune disease before administration of the biologics.

In at least one embodiment of the present disclosure, the biologics may include an antibody drug, a disease-modifying antirheumatic drug, and/or selective immune inhibitor.

In at least one embodiment of the present disclosure, the selective immune inhibitor may include TNF inhibitor, CTLA-4 inhibitor, and/or CD20 antagonist.

In at least one embodiment of the present disclosure, the immune response may include a predicted gene expression data of the second patient with autoimmune disease after administration of the biologics.

In at least one embodiment of the present disclosure, the gene expression data may include a gene expression profile, a differential expression gene data, and/or a biological pathway data.

In at least one embodiment of the present disclosure, the genetic feature extraction unit may be further used to provide a third transcriptomic data of the single cell to the deep learning model to create a testing data set; and the immune response prediction model may be further used to import the third transcriptomic data therein to predict the immune response.

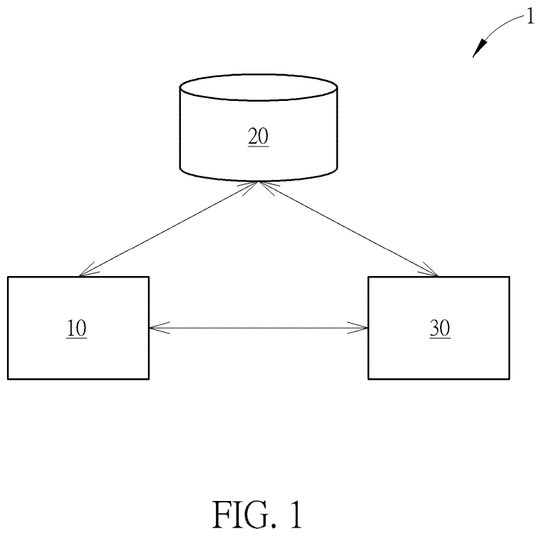

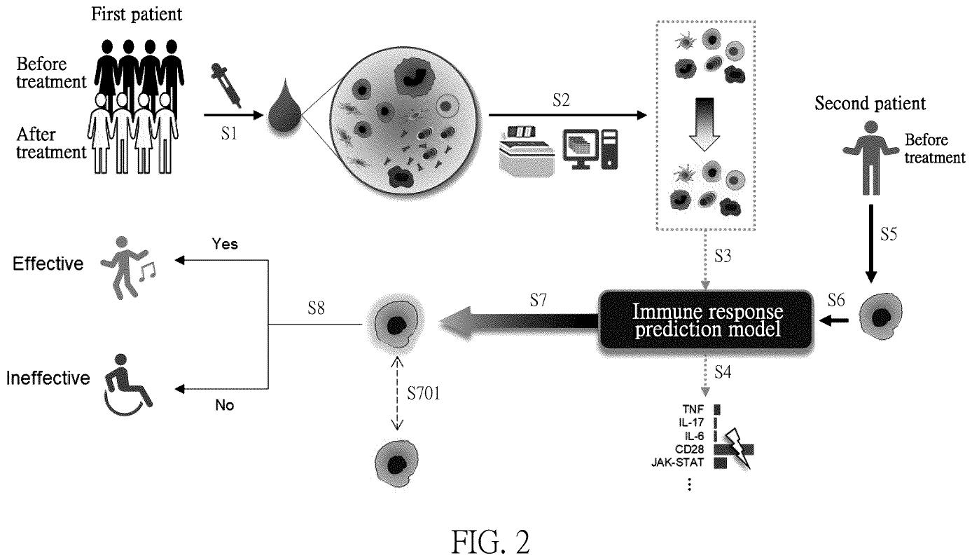

Referring to FIG. 1, a system 1 for predicting an immune response is illustrated, including: a genetic feature extraction unit 10, a data storage unit 20, and a model building and optimization unit 30. Described elements of the system 1 may be connected to each other via any suitable wired or wireless means, of which the present disclosure is not limited thereto.

In some embodiments, the elements of the system may be individually realized as any suitable computing device, apparatus, program, system, or the like, but the present disclosure is not limited thereto. For example, any two or three of the genetic feature extraction unit 10, the data storage unit 20 and the model building and optimization unit 30 may be integrated instead of being realized as three distinct units. In some embodiments, said three elements may also be integrated and realized in a cloud computing environment. Nevertheless, without straying from the operation philosophy of the present disclosure, the configuration of said elements of the system may be realized in any suitable forms and should not be restrictive to the scope of the present disclosure.

Referring to FIG. 2, a method for predicting an immune response is illustrated, and the operation process is denoted as arrows (described as “step(s)” herein) and explained herefrom.

In some embodiments, step S1 denotes that a single-cell transcriptomic data of blood samples obtained from a first patient with autoimmune disease before and after biologics treatment are collected.

As used herein, the terms “first transcriptomic data” and “second transcriptomic data” are intended to refer to the single-cell transcriptomic data obtained from a first patient with autoimmune disease before and after biologics treatment, respectively.

In some embodiments, step S2 denotes that the aforementioned single-cell transcriptomic data of the blood samples undergo data preprocessing and data transformation processing, e.g., single-cell RNA sequencing (scRNA-seq).

In some embodiments, step S3 denotes that a first genetic feature of the first transcriptomic data and a second genetic feature of the second transcriptomic data are obtained by the aforementioned data preprocessing and data transformation processing as a training data set for training and/or building an immune response prediction model. The second genetic feature of the second transcriptomic data may be, but not limited to, genetic feature of immune perturbation.

In some embodiments, step S4 denotes the aforementioned immune response prediction model is trained to be able to identify the first genetic feature of the first transcriptomic data and the second genetic feature of the second transcriptomic data, e.g., the perturbed pathway of the second transcriptomic data.

In some embodiments, step S5 denotes that a single-cell transcriptomic data of blood samples obtained from a second patient with autoimmune disease before biologics treatment is collected.

As used herein, the term “third transcriptomic data” is intended to refer to the single-cell transcriptomic data obtained from a second patient with autoimmune disease before biologics treatment.

In some embodiments, step S6 denotes that a third genetic feature of the third transcriptomic data as the testing data set is imported to the aforementioned immune response prediction model.

In some embodiments, steps S7 and S8 denote that the aforementioned immune response prediction model predicts an immune response (e.g., to evaluate if the target pathway is perturbed) of the second patient after the biologics treatment based on the testing data in order to predict the effectiveness of the biologics treatment.

In some embodiments, step S701 denotes that the results of the immune response prediction generated by the aforementioned immune response prediction model may be compared with the real treated results to identify the similarity between the two results.

In some embodiments, the immune response prediction model of the present disclosure may be integrated with a blood sensor hardware device to establish a real-time single-cell blood biologics evaluator for physicians'/medical institution's and/or biologics suppliers' reference.

In some embodiments, the transcriptomic data (e.g., transcriptomic profile) in response to biologics treatment (e.g., anti-CTLA-4 therapy) is captured by the immune response prediction model (e.g., machine-learning based model) of the present disclosure.

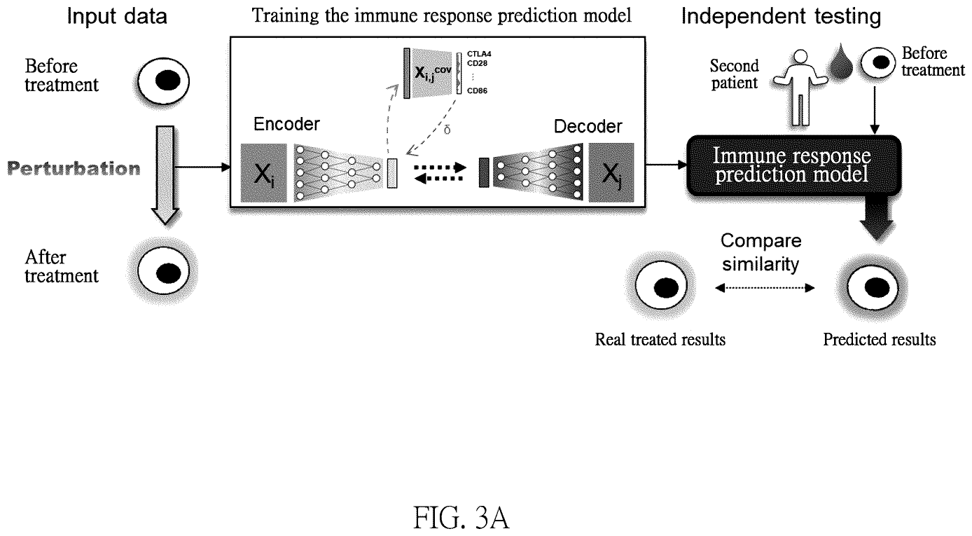

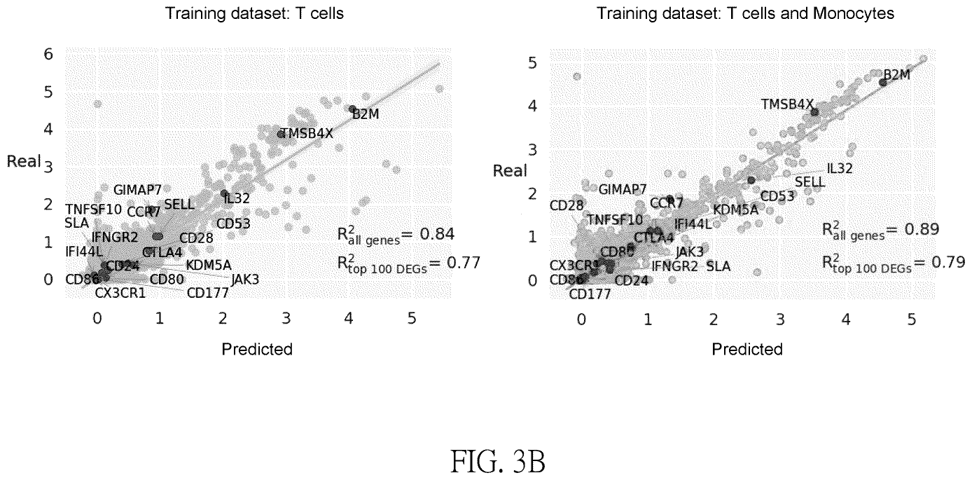

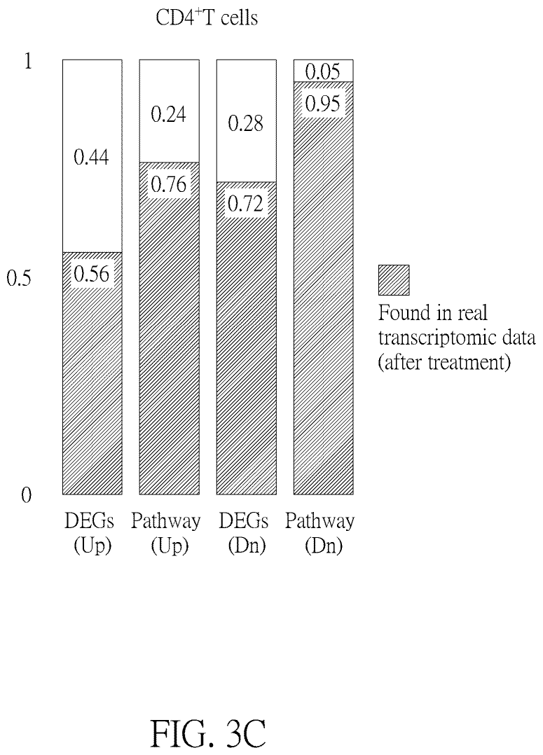

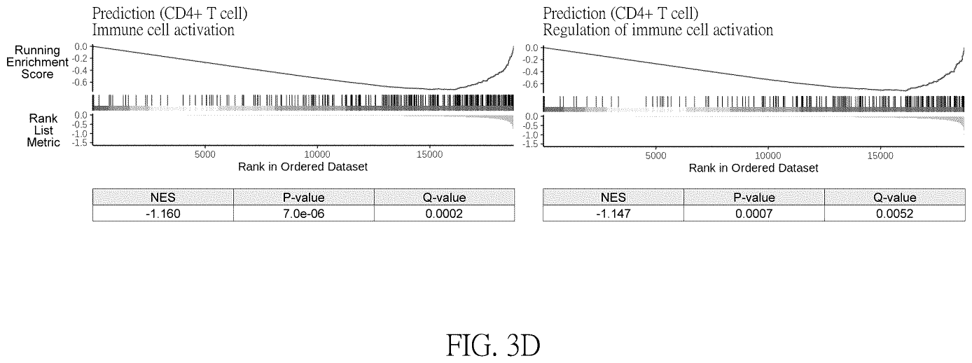

Based on the findings from blood-synovial-shared disease features and blood bDMARD response, blood immune transcriptomic perturbation does not only reflect rheumatoid arthritis (RA) synovial immune phenotypes but also mirror therapeutic efficacies of biologic agents. This phenomenon reveals that transcriptomic perturbation of blood immune cell population may have the potentiality to be compressed by a data-driven learning model and further extrapolate underlying pathways related to disease mechanism. With an understanding of disease mechanism of RA, the transcriptomic data (e.g., transcriptomic profile) of monocytes and T cells are crucial for the model learning and should be included in the training dataset. Meanwhile, given the mechanism action of the biologics treatment (e.g., anti-CTLA-4 therapy), transcriptomic perturbations of CD4⁺ T cells mirror therapeutic efficacies the most and need to be considered as prediction targets. The blood transcriptomic data (e.g., transcriptomic profile) of the first patient before biologics treatment (i.e., the untreated RA patients) and those receiving the biologics treatment (e.g., anti-CTLA-4 therapy) as input data are utilized to train the immune response prediction model of the present disclosure. Therefore, where the blood transcriptomic data of the second patient before treatment is provided to the immune response prediction model of the present disclosure, the model may predict gene expression data (e.g., gene expression profiles) of treated CD4⁺ T cells of the second patient excluded from the training dataset. Model performances were evaluated by similarity (Pearson correlation coefficient, R²) between the predicted transcriptomic data and real treated transcriptomic data (i.e., CD4⁺ T cell transcriptomic profiles) (FIG. 3A).

In some embodiments, the immune response prediction model of the present disclosure may be, but not limited to, a variational autoencoder composed of an encoder and a decoder (FIG. 3A).

To assess the impact of monocytes in response to the biologics treatment (i.e., anti-CTLA-4 therapy), two separate training datasets comprised of T cells (including naive CD4⁺ T cells, CD4⁺ memory T cells, CD8⁺ T cells, central memory T cells, and effector memory T cells) (left panel of FIG. 3B) and T cells and monocytes (including classical monocytes and non-classical monocytes) (right panel of FIG. 3B) for model training, respectively, are designed and compared prediction performances thereof. FIG. 3B shows the performances of the immune response prediction models with different training datasets for predicting the immune response (e.g., transcriptomic profiles) of CD4⁺ T cells of the second patient with unknown therapeutic outcome (i.e., Disease Activity Score-28 (DAS28) improvement undetermined) after biologics treatment (i.e., anti-CTLA-4 treatment). The gene expression of untreated RA features (e.g., CCR7, CX3CR1, GIMAP7, IFNGR2, TNFSF10, CD53, SLA, and CD177) and anti-CTLA-4 therapy effects (e.g., B2M, TMSB4X, IFI44L, KDM5A, JAK3, SELL, IL32, and CD24) are highlighted and utilized to examine the model accuracies. The immune response prediction model trained by T cells and monocytes outperformed that trained by T cells by having greater transcriptomic similarities of real treated transcriptomic data on all 38,300 genes (R²=0.89), top-100 differentially expressed genes (DEGs) (R²=0.79) and highlighted genes (dark dots). Therefore, the model (i.e., the aforementioned VAE+Vec model) trained by T cells and monocytes is determined as immune response prediction model of the present disclosure.

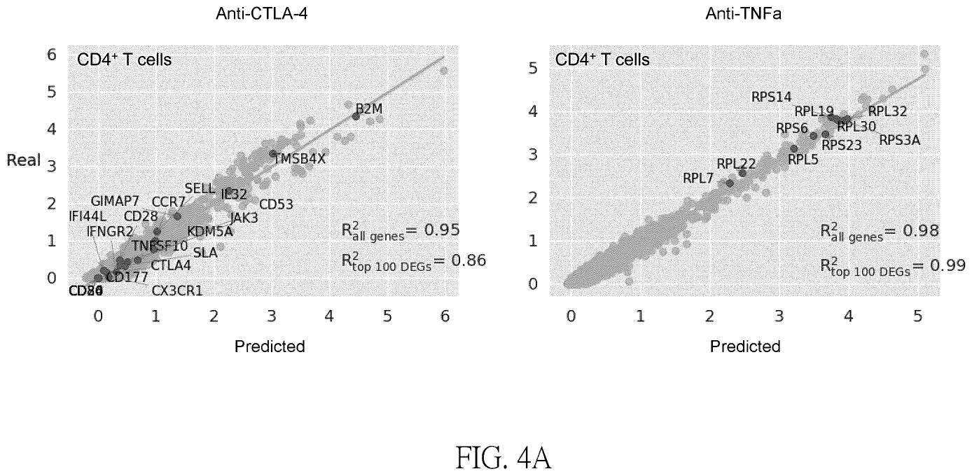

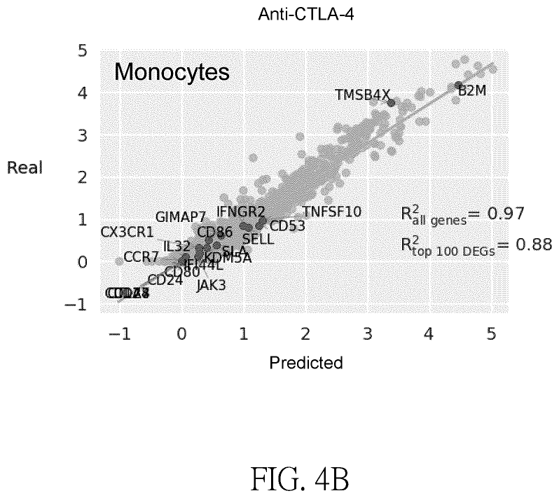

Furthermore, training datasets comprised CD4⁺ T cells or monocytes in response to the biologics treatment (i.e., anti-CTLA-4 treatment or anti-TNF (tumor necrosis factor α) treatment) only are also provided for the model training to access the prediction performances. FIG. 4A and FIG. 4B show the performances of the immune response prediction models for predicting the immune response (e.g., transcriptomic profiles) of CD4⁺ T cells (FIG. 4A) or monocytes (FIG. 4B) of the second patient with unknown therapeutic outcome after biologics treatment (i.e., anti-CTLA-4 treatment (FIG. 4A and FIG. 4B) or anti-TNF treatment (FIG. 4A)). The gene expression of untreated RA features and anti-CTLA-4 treatment or anti-TNF treatment effects are highlighted and utilized to examine the model accuracies. The immune response prediction model trained by CD4⁺ T cells or monocytes has high performance with great transcriptomic similarities of real treated transcriptomic data on all 38,300 genes (R²=0.95; R²=0.98; R²=0.97), top-100 differentially expressed genes (DEGs) (R²=0.86; R²=0.99; R²=0.88) and highlighted genes (dark dots).

Apart from predicted transcriptomic data (e.g., transcriptomic profiles), the DEG-pathway similarities between predicted transcriptomic data and real transcriptomic data is inspected by comparing to the CD4⁺ T cells predicted transcriptomic data of the second patient (i.e., predicted transcriptomic data of the untreated RA patient after biologics treatment). According to the results from model-predicted transcriptomic data, there are 56% up-regulated DEGs, 76% up-regulated pathways, 72% down-regulated DEGs and 95% down-regulated pathways are identical to those from real transcriptomic data (FIG. 3C; “Up” stands for “up-regulated” and “Dn” stands for “down-regulated”).

Referring to FIG. 3D, visualizing the distribution of the gene set of immune cell activation and regulation of immune cell activation, and the enrichment score is plotted by comparing the predicted transcriptomic data (e.g., predicted transcriptomic profiles) (i.e., predicted post-treatment data) and real pre-treatment transcriptomic data (e.g., real pre-treatment transcriptomic profiles). The significance is determined from P-values (≤0.05) provided from the gene set enrichment analysis (GSEA). The upper part of both left and right panel of FIG. 3D illustrates the running enrichment score of the gene set. In the middle part of both left and right panel of FIG. 3D, the black vertical bars show the position in the ranked list of genes; the horizontal colored bars show a positive (red) and negative (blue) enrichment score; and a plot illustrates the ranked list metric of pathway gene sets based on the t-test. Normalized enrichment score (NES), P-value, and Q-value provided from the GSEA are listed in the table shown on the lower part of both left and right panel of FIG. 3D. The blood-synovial-shared anti-CTLA-4-perturbed pathways may be observed by comparing predicted transcriptomic data (e.g., predicted transcriptomic profiles) (i.e., predicted post-treatment data) and real pre-treatment transcriptomic data (e.g., real pre-treatment transcriptomic profiles).

In some embodiments, the immune response prediction model of the present disclosure may not only reflect cellular post-treatment transcriptomic data but also capture the crucial suite of perturbed genes and simulate same pathway perturbation.

FIG. 5A, FIG. 5B, FIG. 5C, and FIG. 5D show the targeted cellular pathways of CD4⁺ T cells reflected treatment-response stratification.

As shown in FIG. 5A, a flowchart demonstrating if the immune response prediction model of the present disclosure may reflect biologics-treated (e.g., anti-CTLA-4-treated) transcriptomic features of CD4⁺ T cells from responders (Rs) and non-responders (NRs).

In some embodiments, the terms “responders (Rs)” and “non-responders (NRs)” used herein are intended to refer the patients who are or are not expected to respond to the biologics treatment, respectively, by prediction performed via the immune response prediction model of the present disclosure.

Referring to FIG. 5B, visualizing the distribution of the gene set of therapeutic pathway and the enrichment score is plotted by comparing the real data (e.g., real transcriptomic data) of responders and non-responders via the immune response prediction model of the present disclosure (demonstrated in the upper part of FIG. 5B). The significance is determined from q-value (i.e., Benjamini-Hochberg adjusted p-value) provided from the gene set enrichment analysis. The plots contained in the dotted line of FIG. 5B illustrates the running enrichment score of the gene set of mRNA metabolic process, amide biosynthetic process and peptide metabolic process; the black vertical bars show the position in the ranked list of genes; and the horizontal colored bars show a positive (red) and negative (blue) enrichment score. Q-value, rank, leading edge signal, and gene set size are provided from the gene set enrichment analysis are shown on the upper part of FIG. 5B. Overlapping pathways identified the distinguished therapeutic pathways, e.g., pathways related to mRNA metabolic process, amide biosynthetic process, and peptide metabolic process, between Rs and NRs with reverse regulation of each other and filters of q-value, rank, leading-edge signaling, and gene set size. Under biologics treatment (e.g., anti-CTLA-4 treatment) perturbation, in real CD4⁺ T cells of the Rs (e.g., four Rs), GSEA pathways of mRNA metabolic process are down-regulated, and both amide biosynthetic process and peptide metabolic process are up-regulated, and vice versa for the NRs (e.g., three NRs).

In some embodiments, the term “GO pathways” stands for Gene Ontology pathways.

Referring to FIG. 5C, visualizing the distribution of the gene set of therapeutic pathway and the enrichment score is plotted by comparing the predicted data (e.g., predicted transcriptomic data) of responders and non-responders via the immune response prediction model of the present disclosure (demonstrated in the upper part of FIG. 5B). The significance is determined from q-value (i.e., Benjamini-Hochberg adjusted p-value) provided from the GSEA. The plots illustrates the running enrichment score of the gene set of mRNA metabolic process, amide biosynthetic process and peptide metabolic process; the black vertical bars show the position in the ranked list of genes; and the horizontal colored bars show a positive (red) and negative (blue) enrichment score. The same pathway perturbations in FIG. 5B are all identified from predicted data (e.g., predicted transcriptomic data) generated by the immune response prediction model of the present disclosure.

Referring to the upper part of FIG. 5D, the target pathway perturbations of the Rs and NRs predicted by the immune response prediction model of the present disclosure are shown individually by heat maps of normalized enrichment score (NES). The bottom left of FIG. 5D shows that the patient (i.e., the patient TP-01 in validation group) labeled a responder shows the target pathway perturbations equivalent to the Rs in predicted data. The bottom center and bottom right of FIG. 5D shows that the untreated RA patients without therapeutic outcomes are stratified by their NES of biologics treatment (e.g., anti-CTLA-4 treatment) perturbed target pathways predicted by the immune response prediction model of the present disclosure from public datasets. Five of these 23 patients (i.e., the patients TP-02, TP-03, TP-04, TP-05, and TP-06 in unknown group) are classified as non-responders based on their pathway perturbations similar to NRs in predicted data.

In some embodiments, the term “DP” stands for discovery patient; and the term “TP” stands for testing patient. In some embodiments, discovery patient is a patient who responds to the anti-CTLA-4 treatment (responder) or does not respond to the anti-CTLA-4 treatment (non-responder). In some embodiments, testing patient is a patient whose response of anti-CTLA-4 treatment has not been determined.

In some embodiments, the discovery groups of four responders are represented as DP-01, DP-02, DP-03, and DP-04; the discovery groups of three non-responders are represented as DP-05, DP-06, and DP-07; the patient in validation group labeled as a responder is represented as TP-01; and the patients in unknown group labeled as a non-responder are represented as TP-02, TP-03, TP-04, TP-05, and TP-06.

Regarding the advance of the present disclosure described above, given that the global trend of shifting towards treatments based on individual patient profiles, the present disclosure provides a method and system to predict individual immune responses, enabling tailored therapies (e.g., genomic-based personalized cancer treatments). Moreover, using big data and artificial intelligence (AI) to streamline drug development and focusing on better patient stratification to improve trial outcomes are also the global trend in the technical field of the present disclosure. Therefore, the present disclosure may also provide an application of AI for efficient drug trial design and patient selection (e.g., AI platforms used by pharmaceutical companies for drug discovery) and improve the patient selection, enhancing trial success rates (e.g., use of biomarkers in selecting trial participants for targeted therapies).

Regarding the advance of the present disclosure described above, given that the global trend of emphasizing on data-driven evidence for drug approval since FDA's approval processes increasingly relying on detailed clinical data, the present disclosure provides a detailed data for regulatory compliance and safety. Moreover, reducing healthcare costs while improving outcomes is also the global trend in the technical field of the present disclosure. Therefore, the present disclosure may also contribute to cost-effective healthcare (e.g., use of telemedicine to reduce healthcare delivery costs).

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for predicting an immune response, comprising:
a genetic feature extraction unit providing a transcriptomic data of a single cell to a deep learning model to create a training data set; and
a model building and optimization unit building an immune response prediction model based on the training data set.

2. The method of claim 1, wherein the single cell comprises an immune cell.

3. The method of claim 2, wherein the immune cell comprises a monocyte, a lymphocyte, and/or a granulocyte.

4. The method of claim 3, wherein the lymphocyte comprises a T cell.

5. The method of claim 1, wherein the transcriptomic data provided to the deep learning model comprises a first transcriptomic data and a second transcriptomic data, and the immune response prediction model is built by training a variational autoencoder of the deep learning model with the first transcriptomic data and the second transcriptomic data.

6. The method of claim 5, further comprising:
providing a third transcriptomic data of the single cell to the deep learning model to create a testing data set; and
importing the third transcriptomic data to the immune response prediction model to predict the immune response.

7. The method of claim 6, wherein
the providing the transcriptomic data of the single cell to the deep learning model to create the training data set comprises:
using a first genetic feature of the first transcriptomic data and a second genetic feature of the second transcriptomic data as the training data set by data preprocessing and data transformation processing; and
the providing the third transcriptomic data of the single cell to the deep learning model to create the testing data set comprises:
using a third genetic feature of the third transcriptomic data as the testing data set.

8. The method of claim 7, wherein the data preprocessing comprises a transcriptomic identification of the single cell, and the data transformation processing comprises a transformation operation of the genetic signature.

9. The method of claim 6, wherein
the first transcriptomic data is obtained from a first blood sample of a first patient with autoimmune disease before administration of a biologics;
the second transcriptomic data is obtained from a second blood sample of the first patient with autoimmune disease after administration of the biologics; and
the third transcriptomic data is obtained from a third blood sample of the second patient with autoimmune disease before administration of the biologics.

10. The method of claim 9, wherein the biologics comprises an antibody drug, a disease-modifying antirheumatic drug, and/or selective immune inhibitor.

11. The method of claim 10, wherein the selective immune inhibitor comprises TNF inhibitor, CTLA-4 inhibitor, and/or CD20 antagonist.

12. The method of claim 9, wherein the immune response comprises a predicted gene expression data of the second patient with autoimmune disease after administration of the biologics.

13. The method of claim 12, wherein the gene expression data comprises a gene expression profile, a differential expression gene data, and/or a biological pathway data.

14. A system for predicting an immune response, comprising:
a genetic feature extraction unit configured to provide a transcriptomic data of a single cell to a deep learning model to create a training data set; and
a model building and optimization unit configured to build an immune response prediction model based on the training data set.

15. The system of claim 14, wherein the single cell comprises an immune cell.

16. The system of claim 15, wherein the immune cell comprises a monocyte, a lymphocyte, and/or a granulocyte, and the lymphocyte comprises a T cell.

17. The system of claim 14, wherein the transcriptomic data comprises a first transcriptomic data and a second transcriptomic data, and the immune response prediction model is built by training a variational autoencoder of the deep learning model with the first transcriptomic data and the second transcriptomic data.

18. The system of claim 17, wherein
the genetic feature extraction unit is further configured to provide a third transcriptomic data of the single cell to the deep learning model to create a testing data set; and
the immune response prediction model is further configured to import the third transcriptomic data therein to predict the immune response.

19. The system of claim 18, wherein the first transcriptomic data is obtained from a first blood sample of a first patient with autoimmune disease before administration of a biologics; the second transcriptomic data is obtained from a second blood sample of the first patient with autoimmune disease after administration of the biologics; and the third transcriptomic data is obtained from a third blood sample of the second patient with autoimmune disease before administration of the biologics.

20. A computer readable medium storing a computer executable code which, when being executed, causes the method of claim 1 to be implemented.
